(19) United States
(12) Patent Application Publication
(10) Pub. No.: US 2005/0227884 A1
(43) Pub. Date: Oct. 13, 2005

(54) Method and composition for removing waxy soils

(75) Inventors: Bryan A. Maser, Inver Grove Heights, MN (US); Denise M. Chandler, St. Paul, MN (US); Tareasa L. Bradley, Shakopee, MN (US)

Correspondence Address: ECOLAB INC., MAIL STOP ESC-F7, 655 LONE OAK DRIVE, EAGAN, MN, 55121, US

(73) Assignee: ECOLAB INC., St. Paul, MN (US)

(21) Appl. No.: 11/097,847

(22) Filed: Apr. 1, 2005

Related U.S. Application Data
(60) Provisional application No. 60/560,102, filed on Apr. 7, 2004.

(51) Int. Cl. A61K 7/025; A61K 7/02

(52) U.S. Cl. 510/136

(57) ABSTRACT

The invention generally pertains to a method and composition for removing waxy soils. The invention also pertains to a method and composition for removing lip cosmetic soils such as lipsticks, Chapsticks™, and the like. The invention further pertains to a method and composition for removing lip cosmetic soils from the surface of drinkware. Finally, the invention pertains to a method and composition for removing lip cosmetic soils from the surface of drinkware wherein the drinkware is first pretreated with an effective amount of a pretreating composition and the drinkware is then treated with a normal washing process. In an embodiment, the drinkware is first pretreated with an effective amount of a pretreating composition to remove substantially all of the lip cosmetic soil from the drinkware. In an embodiment, the drinkware is first pretreated with an effective amount of a pretreating composition to loosen the lip cosmetic soil.

### CROSS REFERENCE TO RELATED APPLICATION

This application claims priority under 35 U.S.C. §119(e) to U.S. Provisional Application entitled “METHOD AND COMPOSITION FOR REMOVING WAXY SOILS”, Ser. No. 60/560,102, filed on Apr. 7, 2004, which is incorporated by reference herein in its entirety.

### FIELD OF THE INVENTION

The invention generally pertains to a method and composition for removing waxy soils. The invention also pertains to a method and composition for removing lip cosmetic soils such as lipsticks, Chapsticks™, and the like. The invention further pertains to a method and composition for removing lip cosmetic soils from the surface of drinkware. Finally, the invention pertains to a method and composition for removing lip cosmetic soils from the surface of drinkware wherein the drinkware is first pretreated with an effective amount of a pretreating composition and the drinkware is then treated with a normal washing process. In an embodiment, the drinkware is first pretreated with an effective amount of a pretreating composition to remove substantially all of the lip cosmetic soil from the drinkware. In an embodiment, the drinkware is first pretreated with an effective amount of a pretreating composition to loosen the lip cosmetic soil.

### BACKGROUND

In restaurants and bars, drinkware, and the top portion of the drinkware in particular, often becomes soiled from lip cosmetics that rub off a patron's lips and onto the drinkware as the patron drinks out of the glass. The lip cosmetic soil is typically very difficult to remove because of the waxy consistency of lip cosmetics. Recently, lip cosmetic soils have become even more difficult to remove as a result of advances in the lip cosmetic industry such as new “long-wearing” lipsticks.

In the past, drinkware have been run through various washing processes depending on the particular method used. These processes are often ineffective at removing the lip cosmetic soil. The result is that the “cleaned” glass still has a lip cosmetic soil on it which is unsettling for the next patron that receives that glass or cup. While any soil remaining on the drinkware may be “clean” in that the soil does not contain any bacteria or other contaminants, the presence of a visual soil gives the next patron the impression that the drinkware is not clean. This in turn reflects poorly on the restaurant or bar.

It is against this background that the present invention has been made.

### SUMMARY

Surprisingly, it has been discovered that pretreating drinkware with an effective amount of a pretreating composition to remove substantially all of the lip cosmetic soil or loosen the lip cosmetic soil prior to running the drinkware through a normal wash cycle, substantially improves the removal of the lip cosmetic soil. The same result can be achieved when only the portion of the drinkware near the rim is pretreated with the pretreating composition.

These and other embodiments will be apparent to those of skill in the art and others in view of the following detailed description of some embodiments. It should be understood, however, that this summary, and the detailed description illustrate only some examples of various embodiments, and are not intended to be limiting to the invention as claimed.

### BRIEF DESCRIPTION OF THE DRAWINGS

FIG. 1 shows an embodiment of a pretreating apparatus wherein the drinkware is pretreated by inverting the drinkware and dipping it into the pretreating apparatus containing the pretreating composition.

FIG. 2 shows a sideview of an embodiment of a pretreating apparatus having a screen on springs.

FIG. 3 shows an embodiment of a pretreating apparatus having a source of pretreating composition and a means for dispensing the pretreating composition into the pretreating apparatus.

FIG. 4 shows an embodiment of a pretreating apparatus with a tension rod.

### DETAILED DESCRIPTION OF SOME EMBODIMENTS

As discussed above, the invention generally relates to a method and composition for removing waxy soils, and lip cosmetic soils in particular, from drinkware. In one embodiment, the drinkware is pretreated with a pretreating composition and then cleaned and sanitized using the normal cleaning process. In one embodiment, the entire drinkware is pretreated. In another embodiment, only the upper portion of the drinkware near the rim of the drinkware is pretreated. In another embodiment, the invention pertains to a device for pretreating the drinkware.

Definitions

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

Weight percent, percent by weight, % by weight, wt %, and the like are synonyms that refer to the concentration of a substance as the weight of that substance divided by the weight of the composition and multiplied by 100.

The recitation of numerical ranges by endpoints includes all numbers subsumed within that range (e.g. 1 to 5 includes 1, 1.5, 2, 2.75, 3, 3.80, 4 and 5).

As used in this specification and the appended claims, the singular forms “a,” “an,” and “the” include plural referents unless the content clearly dictates otherwise. Thus, for example, reference to a composition containing “a compound” includes a mixture of two or more compounds. As used in this specification and the appended claims, the term “or” is generally employed in its sense including “and/or” unless the content clearly dictates otherwise.

The use of the terms “antimicrobial” in this application does not mean that any resulting products are approved for use as an antimicrobial agent.

Method of Removing Lip Cosmetic Soils

The present invention relates to a method of removing lip cosmetic soils from drinkware by first pretreating the drinkware with a pretreating composition and then putting the drinkware through a normal wash process.

The present invention can be used to remove a variety of lip cosmetic soils. For example, the present invention can be used to remove the following lip cosmetics: lipsticks including “long-wearing” lipsticks and “8-hour,” “12-hour,” and “16-hour” lipsticks; lip stains; lip glosses; lip balm such as Chapstick™, petroleum jelly; and the like.

Lip cosmetics may be formulated to have different consistencies depending on the intended purpose and desired aesthetics. For example, a lip balm such as Chapstick™ would have very little or no color in comparison to a lipstick in order to make it more gender neutral. Also, a lip gloss preferably has a more glossy appearance than a lipstick due to a more watery consistency. Despite these formula variations, lip cosmetics generally include similar ingredients. Lip cosmetics usually include a hydrophobic component such as an oil, viscous solid, or wax. Lip cosmetics can also include a pigment, preservative, a moisturizer, a sunscreen, a medicine, for example to heal cracked lips, a fragrance, and flavoring. The lip cosmetic may be a one part or a two part composition. For example, a two part lip cosmetic may include a first composition with the pigment and a second moisturizing or glossy composition.

While not wanting to be held to a scientific theory, it is believed that the hydrophobic portion of the lip cosmetic makes a lip cosmetic soil particularly difficult to remove from drinkware. The hydrophobic portion of the lip cosmetic may be an oil, a viscous solid, or a wax, depending on the desired consistency of the final product. For example, a lip gloss that is rolled onto the lips will tend to be more watery than a lip gloss that is applied using a fingertip. Naturally, one would expect the roll on lip gloss to have a higher oil content than a fingertip lip gloss, which would have more solids or waxes. The hydrophobic component of lip cosmetics may be natural or synthetic. The following is a list of non-limiting examples of hydrophobic materials that are found in lip cosmetics: apple (Pyrus Malus) peel wax, avocado (Persea Gratissima) wax, bayberry (Myrica cerifera) wax, beeswax, candelilla (Euphorbia cerifera) wax, canola oil, carnauba (Copernicia cerifera) wax, castor oil, ceresin, cetyl alcohol, cetyl esters, cocoa (Theobroma cacao) butter, coconut (Cocos nucifera) oil, hydrogenated jojoba oil, hydrogenated jojoba wax, hydrogenated microcrystalline wax, hydrogenated rice bran wax, hydrolyzed beeswax, isostearic acid, jojoba butter, jojoba esters, jojoba wax, lanolin oil, lanolin wax, microcrystalline wax, mineral oil, mink wax, montan acid wax, montan wax, olive (Olea europaea) oil, orange (Citrus aurantium dulcis) peel wax, ouricury wax, oxidized beeswax, oxidized microcrystalline wax, ozokerite, palm kernel wax, paraffin, PEG-6 beeswax, PEG-8 beeswax, PEG-12 beeswax, PEG-20 beeswax, PEG-12 carnauba, petrolatum, petroleum jelly, potassium oxidized microcrystalline wax, rice (Oryza sativa) wax, sesame (Sesamum indicum) oil, shea butter (Butyrospermum parkii), shellac wax, spent grain wax, stearic acid, sulfurized jojoba oil, synthetic beeswax, synthetic candelilla wax, synthetic carnauba, synthetic japan wax, synthetic jojoba oil, synthetic wax, and vegetable oil. It is understood that the pretreating compositions of the invention are capable of removing lip cosmetic soils having the hydrophobic materials described above and those not included in the list above.

Lip cosmetic soils accumulate on any type of drinkware surface typically found in any commercial, institutional, or consumer location including restaurants, bars, hospitals, nursing homes, domestic (consumer) homes, airlines, cafeterias in schools and businesses, and the like. The term “drinkware” includes a variety of materials used to make a drinking container including glass, china, ceramic, plastic, porcelain, Corelleware, Melmac, stoneware, copper, aluminum, acrylic, stainless steel, chrome, crystal, and the like. The term “drinkware” refers to any drinking container and includes for example high ball glasses, low ball glasses, wine glasses, mugs, teacups, pint glasses, shot glasses, martini glasses, snifters, pilsner glasses, champagne flutes, water glasses, and the like.

Pretreating Composition

The pretreating composition of the invention may be organic based or water based. An organic based pretreating composition is effective at removing lip cosmetic soils because of the well known principle that “like dissolves like.” Lip cosmetic soils tend to be hydrophobic, or water hating, and are therefore soluble in hydrophobic or organic based compositions. When an organic based pretreating composition is used, in this embodiment, the composition is preferably present in an amount effective to remove substantially all of the lip cosmetic soil or loosen the lip cosmetic soil.

The water based pretreating compositions described herein are equally effective at removing lip cosmetic soils and have several additional benefits that will be discussed herein. When a water based pretreating composition is used, it is preferably present in an amount effective to remove substantially all of the lip cosmetic soil or to loosen the lip cosmetic soil.

The pretreating composition may be a water thin liquid, an emulsion, a solid, a viscous liquid, etc. In some embodiments, it may be beneficial to form the composition as an emulsion because it allows the raw materials or ingredients to be more stable.

Preferred pretreating compositions are those that are effective at removing lip cosmetic soils without leaving a residue or film when used in a warewashing machine and are effective in a variety of settings including high temperature warewashing machines, low temperature warewashing machines, and manual cleaning.

Organic Based Pretreating Compositions

The organic based pretreating compositions of the present invention include at least one organic solvent. Some non-limiting examples of organic solvents include the following: ketones such as acetone or 2,4-pentadione, commercially available from Aldrich Chemical; esters such as soy esters commercially available from Soygold, ethyl acetate commercially available from Aldrich Chemical, and acetate esters such as Exxate 700, commercially available from Exxon Mobile; paraffin hydrocarbons such as Norpar 13, commercially available from Exxon Mobile; glycol ethers such as propylene glycol phenyl ether, ethylene glycol phenol ether, commercially available from Dow Chemical; glycols such as hexylene; terpenes such as D'limonene, commercially available from Florida Chemical; mineral oils such as those commercially available from Witco Corporation; mixtures thereof and the like. The organic based pretreating compositions of the invention can include more than one organic solvent.

While the organic solvents described above are all suitable for use in the organic based pretreating compositions of the invention, it may be preferred to select the particular organic solvent to avoid particular undesirable characteristics. For example, ketones may be undesirable if the flash point is a concern. Esters and glycols may be undesirable due to their reactivity with alkalinity in the sump of the washing device in the washing step. Terpenes may be undesirable because of their high cost and odor. These organic solvents may be used if their undesirable characteristics are not a concern or can be avoided. The preferred organic solvents for use in the present invention are paraffin mineral oil, and isopropyl palmitate.

When used in the present composition, the organic solvent is included from about 0.1 wt. % to about 100 wt. %, from about 20 wt. % to about 100 wt. %, and from about 50 wt. % to about 100 wt. %.

The organic based pretreating composition does not need a source of alkalinity in order to effectively remove a lip cosmetic soil. Accordingly, the organic based pretreating composition may be free or substantially free of a source of alkalinity. However, the organic based pretreating composition of the invention may optionally include a source of alkalinity to provide a soil removing “boost” to the composition if necessary. In such a situation, organic soluble sources of alkalinity may be included in the organic based pretreating composition. Some non-limiting examples of organic sources of alkalinity include: monoethanolamine, triethanolamine, monoisopropanolamine, and the like.

In addition to the organic solvent, the organic based pretreating compositions of the present invention may include additional functional ingredients that improve the efficacy of the composition or impart some additional benefit. Some examples of additional functional ingredients for use in the organic based pretreating composition include surfactants, hydrotropes, dyes, viscosity modifiers, and the like.

Surfactants

The organic based pretreating compositions of the present invention may optionally include a surfactant. The surfactant is preferably soluble in the organic solvent and is preferably low foaming. Some non-limiting examples of suitable surfactants include the following: chloro capped alcohol ethoxylates such as Antarox BL344, commercially available from Rhodia; EO/PO alcohol ethoxylates such as Antarox 17-R-2, commercially available from Rhodia; and EO/PO alcohol ethoxylates such as Pluronic L62, commercially available from BASF.

When a surfactant is included in the organic based pretreating composition, it is present from about 0.1 wt. % to about 10 wt. %, from about 0.5 wt. % to about 7.5 wt. %, and from about 1.0 wt. % to about 5 wt. %.

Hydrotrope

The organic based pretreating compositions of the present invention may optionally include a hydrotrope for bringing the various ingredients into solution. The hydrotrope is preferably soluble in the organic solvent. Some non-limiting examples of suitable hydrotropes include the following: sodium xylene sulfonate, commercially available from Huntsman; sodium salt of nonanoic acid such as Colotrope, commercially available from Celenese; and phosphate ester such as Monotrope 1296, commercially available from Uniquema.

When a hydrotrope is included in the organic based pretreating composition, it is present from about 0.1 wt. % to about 40 wt. %, from about 0.5 wt. % to about 30 wt. %, and from about 1.0 wt. % to about 20 wt. %.

Dye

The organic based pretreating compositions of the present invention may optionally include a dye. The dye is preferably soluble in the organic solvent. Some non-limiting examples of suitable dyes include the following: LX 6694A Pylakrome Dark Blue and LX 1922 Pylakrome Medium Blue, both commercially available from Pylam.

When a dye is included in the organic based pretreating composition, it is present from about 0.0001 wt. % to about 1.0 wt. %, from about 0.0001 wt. % to about 0.1 wt. %, and from about 0.0001 wt. % to about 0.01 wt. %.

Viscosity Modifier

The organic based pretreating compositions of the present invention may optionally include a viscosity modifier. Including a viscosity modifier may allow the pretreating composition to cling to the drinkware for a period of time and thereby improve the effectiveness of the composition. The viscosity modifier is preferably soluble in the organic solvent. Some non-limiting examples of suitable viscosity modifiers include the following: hydroxyethyl cellulose such as Klucel, commercially available from Aqualon; xanthan gum such as Kelzan, commercially available from Kelco; and acrylamide such as Solagum, commercially available from Seppic.

When a viscosity modifier is included in the organic based pretreating composition, it is present from about 0.1 wt. % to about 1.0 wt. %, from about 0.1 wt. % to about 0.5 wt. %, and from about 0.1 wt. % to about 0.3 wt. %.

Water Based Pretreating Compositions

The water based pretreating compositions of the present invention include at least one source of alkalinity. Some non-limiting examples of suitable sources of alkalinity include sodium hydroxide, commercially available from OxyChem; potassium hydroxide, commercially available from Vulcan Chemical; and sodium carbonate, commercially available from FMC Corporation. The water based pretreating compositions of the present invention may include more than one source of alkalinity.

When used in the present composition, the source of alkalinity is preferably included in an amount sufficient to create a pH in the use solution of about 8-14, from about 10-14, and from about 11-14.

In addition to the source of alkalinity, the water based pretreating compositions of the present invention may include additional functional ingredients that improve the efficacy of the composition or impart some additional benefit. Some examples of additional functional ingredients for use in the water based pretreating composition include surfactants, hydrotropes, dyes, viscosity modifiers, and the like.

Surfactants

The water based pretreating compositions of the present invention may optionally include a surfactant. The surfactant is preferably low foaming. Some non-limiting examples of suitable surfactants include the following: chloro capped alcohol ethoxylates such as Antarox BL344, commercially available from Rhodia; EO/PO alcohol ethoxylates such as Antarox 17-R-2, commercially available from Rhodia; and EO/PO alcohol ethoxylates such as Pluronic L62, commercially available from BASF.

When a surfactant is included in the water based pretreating composition, it is present from about 0.1 wt. % to about 10 wt. %, from about 0.5 wt. % to about 7.5 wt. %, and from about 1.0 wt. % to about 5 wt. %.

Hydrotrope

The water based pretreating compositions of the present invention may optionally include a hydrotrope for bringing the various ingredients into solution. Some non-limiting examples of suitable hydrotropes include the following: sodium xylene sulfonate, commercially available from Huntsman; sodium salt of nonanoic acid such as Colotrope, commercially available from Celenese; and phosphate ester such as Monotrope 1296, commercially available from Uniquema.

When a hydrotrope is included in the water based pretreating composition, it is present from about 0.1 wt. % to about 40 wt. %, from about 0.5 wt. % to about 30 wt. %, and from about 1.0 wt. % to about 20 wt. %.

Dye

The water based pretreating compositions of the present invention may optionally include a dye. Some non-limiting examples of suitable dyes include the following: LX 6694A Pylakrome Dark Blue and LX 1922 Pylakrome Medium Blue, both commercially available from Pylam.

When a dye is included in the water based pretreating composition, it is present from about 0.0001 wt. % to about 1.0 wt. %, from about 0.0001 wt. % to about 0.1 wt. %, and from about 0.0001 wt. % to about 0.01 wt. %.

Viscosity Modifier

The water based pretreating compositions of the present invention may optionally include a viscosity modifier. Including a viscosity modifier may allow the pretreating composition to cling to the drinkware for a period of time and thereby improve the effectiveness of the composition. Some non-limiting examples of suitable viscosity modifiers include the following: hydroxyethyl cellulose such as Klucel, commercially available from Aqualon; xanthan gum such as Kelzan, commercially available from Kelco; and acrylamide such as Solagum, commercially available from Seppic.

When a viscosity modifier is included in the water based pretreating composition, it is present from about 0.1 wt. % to about 1.0 wt. %, from about 0.1 wt. % to about 0.5 wt. %, and from about 0.1 wt. % to about 0.3 wt. %.

Builder

The water based pretreating compositions of the present invention may optionally include a builder. Some non-limiting examples of suitable builders include the following: sodium tripolyphosphate, sodium carbonate, sodium perborate, and the like.

When a builder is included in the water based pretreating composition, it is present from about 0.1 wt. % to about 30 wt. %, from about 0.1 wt. % to about 20 wt. %, and from about 0.1 wt. % to about 15 wt. %.

Washing Process

The method of the present invention includes pretreating drinkware with a pretreating composition. In certain embodiments, the purpose for the pretreating step is to remove substantially all of the lip cosmetic soil from the drinkware. In certain embodiments, the purpose of the pretreating step is to loosen the lip cosmetic soil from the drinkware such that the lip cosmetic soil is substantially removed after the drinkware is put through a wash cycle. Accordingly, the pretreating step can apply the pretreating composition to the entire surface of the drinkware. Alternatively, the pretreating step can apply the pretreating composition to the upper portion of the drinkware near the rim where a lip cosmetic soil is typically located. For example, the pretreating step can apply the pretreating composition to the upper 2 inches of the drinkware, to the upper 1 and a half inches of the drinkware, and to the upper one inch of the drinkware.

The pretreating step can apply the pretreating composition to the drinkware in a variety of ways including spraying, misting, foaming, dipping, fogging, rolling, brushing, wiping (e.g. with an impregnated wipe), applying with a sponge, and the like.

In an embodiment, in order for the pretreating composition to effectively remove substantially all of the lip cosmetic soil, the pretreating composition contacts the drinkware for at least about 5 to 10 seconds, for at least about 5 to 20 seconds, and for at least about 5 to 60 seconds. In an embodiment, in order for the pretreating composition to effectively loosen the lip cosmetic soil, the pretreating composition contacts the drinkware for about 10-30 seconds.

Following the pretreating step, the drinkware is then washed in a normal wash process. For example, the drinkware may be washed manually. The drinkware may also be washed in a warewashing machine including a door or hood machine, a conveyor machine, a Flight machine, or a rotating bar glass washer.

The method of the present invention may be carried out anywhere that drinkware may become soiled including any commercial, institutional, or consumer location such as restaurants, bars, hospitals, nursing homes, domestic (consumer) homes, airlines, cafeterias in schools and businesses, and the like. The method of the present invention may be carried out in the front of the house, for example, at the bar, or in the back of the house, for example in the kitchen.

When using the method of the present invention it may be beneficial to carry out the pretreating step in conjunction with a pretreating apparatus for applying the pretreating composition. For example, it may be beneficial to use the pretreating step in conjunction with a pretreating apparatus containing the pretreating composition such that the pretreating composition may be applied to the drinkware by placing the drinkware in the pretreating apparatus. The pretreating apparatus and its various embodiments are described in detail below with reference to the figures. When referring to the figures, like structures and elements shown throughout are indicated with like reference numerals.

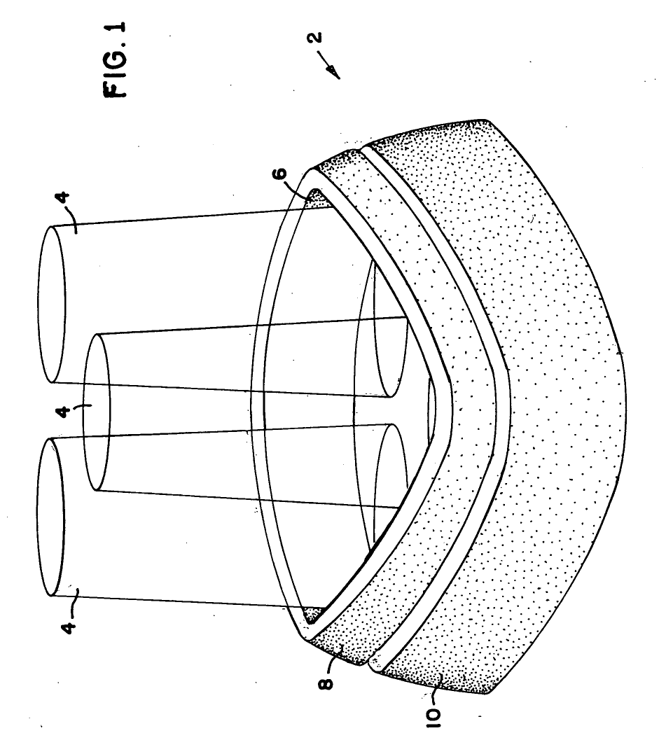

FIG. 1 shows an apparatus (2) for applying the pretreating composition (6) by inverting the drinkware (4) and dipping the drinkware (4) into the apparatus (2). The apparatus (2) includes a container (10) for holding the pretreating composition (6). The apparatus (2) may optionally include padding (8) to prevent chipping or breaking of the drinkware (4).

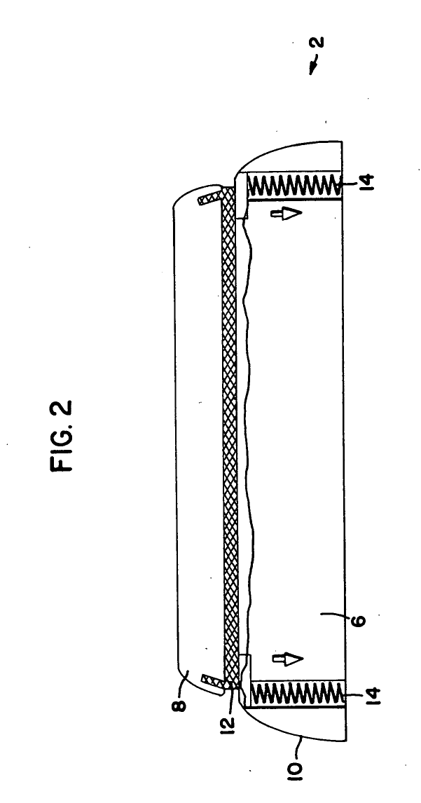

In certain embodiments, shown in FIG. 2, the apparatus (2) may include a screen (12), such as a mesh or plastic screen, for straining any solid particles from the drinkware (4). In certain embodiments, the apparatus (2) may include a screen (12) on springs (14) where the screen (12) normally sits above the pretreating composition (6) and the screen (12) is lowered into the pretreating composition (6) when drinkware (4) is placed on the screen (12) or when the user depresses drinkware (4) on the screen (12).

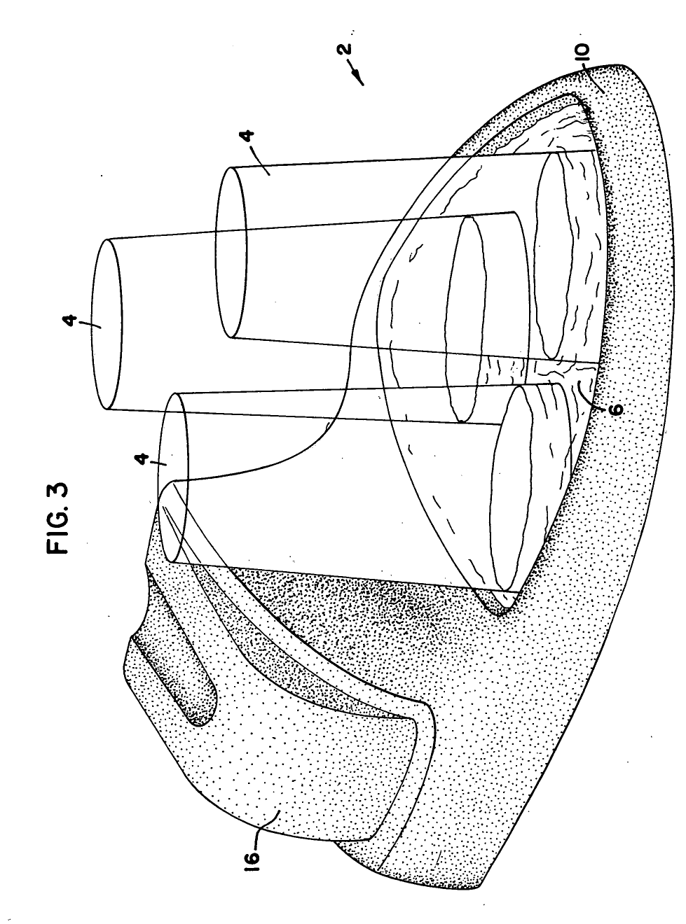

In certain embodiments, shown in FIG. 3, the apparatus (2) may include a source of pretreating composition (6) as well as means for dispensing the pretreating composition (16) into the apparatus (2). For example, the pretreating composition may be dispensed using a gravity fed dispenser or a vacuumized pump such as a piston pump or a peristaltic pump. The pretreating composition may also be dispensed using a positive displacement pump. The pump may be located on the pretreating composition package. The actual pretreating composition may be located anywhere. For example, the pretreating composition may be located near the dispenser, for example, in a docking station near the dispenser, or the pretreating composition may be located at a remote location.

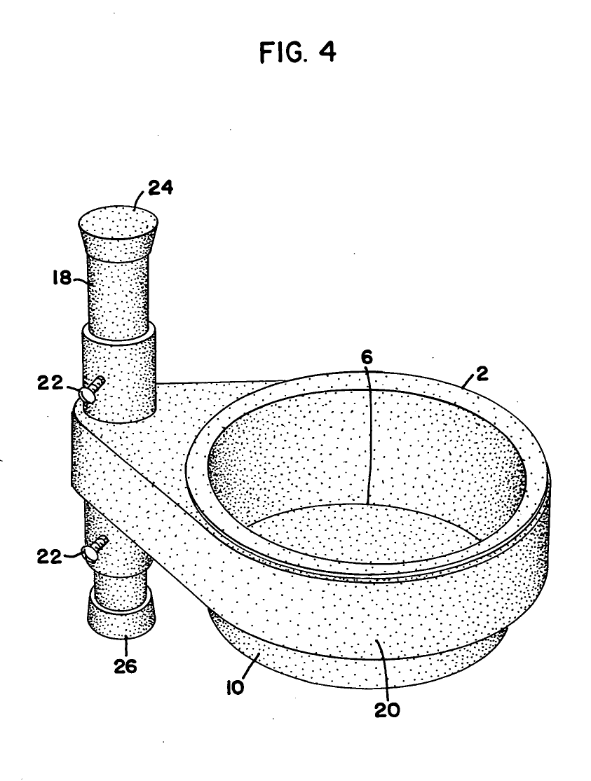

In certain embodiments, shown in FIG. 4, the apparatus (2) may include an attachment means such as a tension rod (18) for attaching the apparatus to another surface, for example the sink, the wall, underneath the bar counter, on or inside the warewashing machine, near the dirty glass prepping area, and the like. The apparatus (2) may include a container (10) and a holder (20) for holding the container on the tension rod (18). The tension rod (18) may include a first end (24) and a second end (26) and one or more fasteners (22) for locking the tension rod (18). Such an apparatus is particularly useful for front of the house applications where space is limited and it is desirable to keep the apparatus out of the view of the patrons, for example, under the bar counter.

In certain embodiments, the apparatus may include a self-filling feature where the apparatus is automatically refilled when it gets too low, for example by a float that triggers a switch when the pretreating composition falls below a certain level. In certain embodiments, the apparatus includes a drain. The drain is particularly useful when used in conjunction with an organic based pretreating composition because it allows the water from a beverage or ice to separate from the organic pretreating composition to the bottom of the apparatus and drain off. In certain embodiments, the apparatus includes padding on the inside and/or outside rim of the apparatus to prevent the user from chipping the drinkware. In certain embodiments, the apparatus may include a sponge, brush, or other physical structure for providing mechanical action to assist in removing lip cosmetic soil from glassware, although it is understood that the pretreating composition alone is effective.

The apparatus may be any shape.

The apparatus may be designed to hold any number of glasses.

For a more complete understanding of the invention, the following examples are given to illustrate some embodiment. These examples and experiments are to be understood as illustrative and not limiting. All parts are by weight, except where it is contrarily indicated.

### EXAMPLES

The following chart provides a brief explanation of certain chemical components used in the following examples:

TABLE 1
Trade Names and Corresponding Descriptions of Some Chemicals
Used in the Examples

| Trademark/ | | |
|---|---|---|
| Chemical Name | Description | Provider |
| 2,4-pentadione | Organic Solvent | Aldrich Chemical |
| Acetone | Organic Solvent | Aldrich Chemical |
| Soygold 1000 | Soy Ester Organic Solvent | Soygold |
| Ethylacetate | Ester | Aldrich Chemical |
| Exxate 700 | | Exxon Mobile |
| Norpar 13 | Paraffin Organic Solvent | Exxon Mobile |
| Propylene Glycol Phenyl | Organic Solvent | Dow Chemical |
| Ether | | |
| Ethylene Glycol Phenol | Organic Solvent | Dow Chemical |
| Ether | | |
| D'limonene | Terpene | Florida Chemical |
| Mineral Oil | Organic Solvent | Witco Corp. |

### Example 1

Example 1 compared the efficacy of various organic solvents at removing lip cosmetic soils from a regular water glass. A water glass was soiled with Really Rose 740, a lipstick commercially available from L'Oreal's Endless line of lipsticks, by applying the lipstick to a rubber glove on a thumb and then pressing the thumb near the rim of the water glass to simulate a lip cosmetic soil from lips. The water glass was then dipped into the organic solvent. The glass has approximately 0.5 grams of solvent on the glass. After dipping the glass, the glass was put through a wash cycle in an ET-1 rotating glass washer from Jackson Inc. During the wash cycle, the glass was exposed to Ultra Klene, a detergent commercially available from Ecolab Inc., Ecosan, a sanitizer commercially available from Ecolab Inc., and Rinse Dry, a rinse aid commercially available from Ecolab Inc. The operating temperature of the dish machine was 120° F. After the glasses were washed in the rotating glass washer, they were visually evaluated and given a lipstick soil removal index on a scale of 1 to 5 for the removal of the lip cosmetic soil where 1 was no lipstick removal and 5 was 100% removal. Table 2 shows the results.

TABLE 2
Lip Cosmetic Soil Removal Using an
Organic Based Pretreating Composition

| Solvent | 1 | 2 | 3 | 4 | 5 |
|---|---|---|---|---|---|
| Ketones | | | | | |
| Acetone | X | | | | |
| Esters | | | | | |
| Soygold 1000 | | | | | X |
| Paraffin | | | | | |
| Norpar 13 | | | | | X |
| Glycol Ethers | | | | | |
| Propylene Glycol Phenol Ether | X | | | | |
| Ethylene Glycol Phenol Ether | X | | | | |
| Terpenes | | | | | |
| D'limonene | | | | | X |
| Mineral Oil | | | | X | |
| Glycols | | | | | |
| Hexylene | | | | | X |

According to Table 2, the esters, paraffin, glycols, terpene, and mineral oil performed the best.

### Example 2

Example 2 looked at the efficacy of a water based pretreating composition at removing a lip cosmetic soil. For this example, a regular water glass was soiled as in Example 1. The glass was dipped in Titan, a detergent composition commercially available from Kay Chemical. The glass was cleaned in a rotating glass washer as in Example 1 and visually evaluated and given a lipstick soil removal index on a scale of 1 to 5 where 1 was no lipstick removal and 5 was 100% lipstick removal. Table 3 shows the results.

TABLE 3
Lip Cosmetic Soil Removal Using a
Water Based Pretreating Composition

| | 1 | 2 | 3 | 4 | 5 |
|---|---|---|---|---|---|
| Titan | | | | X | |

Table 3 shows that the water based pretreating composition performed as well as the organic based pretreating compositions in Example 1.

The foregoing summary, detailed description, and examples provide a sound basis for understanding the invention, and some specific example embodiments of the invention. Since the invention can comprise a variety of embodiments, the above information is not intended to be limiting. The invention resides in the claims.

1. (canceled)

2. (canceled)

3. (canceled)

4. (canceled)

5. A method of removing a lip cosmetic soil from drinkware comprising:
a. providing drinkware having a lip cosmetic soil;
b. placing the drinkware in a pretreating apparatus having a pretreating composition;
c. applying a pretreating composition to at least the upper one inch of the drinkware for at least about 5 seconds, the pretreating composition selected from the group consisting of an organic based pretreating composition and a water based pretreating composition; and
d. washing the drinkware.

6. The method of claim 5, wherein the lip cosmetic soil is selected from the group consisting of lipstick, lip stain, lip gloss, lip balm, petroleum jelly and mixtures thereof.

7. The method of claim 5, wherein the drinkware is selected from the group consisting of high ball glasses, low ball glasses, wine glasses, mugs, teacups, pint glasses, shot glasses, martini glasses, snifters, pilsner glasses, champagne flutes, and water glasses.

8. The method of claim 5, wherein the pretreating apparatus comprises at least one additional feature selected from the group consisting of a screen, a spring, a dispenser, a float, a drain, padding and combinations thereof.

9. The method of claim 5, wherein the drinkware is inverted prior to placing the drinkware in the pretreating apparatus.

10. The method of claim 5, wherein the pretreating composition is applied to at least the upper one and a half inches of the drinkware.

11. The method of claim 5, wherein the pretreating composition is applied to at least the upper two inches of the drinkware.

12. The method of claim 5, wherein the pretreating composition is applied to the entire drinkware.

13. The method of claim 5, wherein the pretreating composition is an organic based pretreating composition.

14. The method of claim 13, wherein the organic based pretreating composition comprises an organic solvent.

15. The method of claim 14, wherein the organic solvent is selected from the group consisting of ketones, esters, hydrocarbons, glycol ethers, glycols, terpenes, mineral oils, and mixtures thereof.

16. The method of claim 13, wherein the organic pretreating composition further comprises an additional functional ingredient selected from the group consisting of surfactants, hydrotropes, dyes, viscosity modifiers, and mixtures thereof.

17. The method of claim 5 wherein the pretreating composition is a water based pretreating composition.

18. The method of claim 17, wherein the water based pretreating composition further comprises an additional functional ingredient selected from the group consisting of surfactants, hydrotropes, dyes, viscosity modifiers, builders, and mixtures thereof.

19. The method of claim 5, wherein the drinkware is manually washed.

20. The method of claim 5, wherein the drinkware is washed in a warewashing machine.

21. The method of claim 5, wherein the pretreating apparatus is located in the front of the house.

22. The method of claim 5, wherein the pretreating apparatus is located in the back of the house.

23. The method of claim 5, wherein the pretreating apparatus is located inside a warewashing machine.

24. A method of removing a lip cosmetic soil from drinkware comprising:
a. providing drinkware having a lip cosmetic soil;
b. placing the drinkware in a pretreating apparatus having a pretreating composition;
c. applying a pretreating composition to at least the upper one inch of the drinkware for at least about 5 seconds, in an amount effective to produce a lipstick soil removal index of at least 4, the pretreating composition selected from the group consisting of an organic based pretreating composition, and a water based pretreating composition; and
d. washing the drinkware.
